(19) United States
(12) Patent Application Publication
(10) Pub. No.: US 2013/0052252 A1
(43) Pub. Date: Feb. 28, 2013

(54) Hard Coated Confectionary Having A Consumable Soft Chewing Core With A Heat Sensitive Particulate Active And Method For Making Same

(71) Applicants: Anthony John Bell, Andover, NJ (US); Shawna Dee Eddy, Charlotte, NC (US); Mark Brian Jones, Cleveland, NC (US); Richard C. Zulman, Cornelius, NC (US)

(72) Inventors: Anthony John Bell, Andover, NJ (US); Shawna Dee Eddy, Charlotte, NC (US); Mark Brian Jones, Cleveland, NC (US); Richard C. Zulman, Cornelius, NC (US)

(21) Appl. No.: 13/218,201

(22) Filed: Aug. 25, 2011

(51) Int. Cl. A61K 9/68; A61K 9/36; A61P 3/02

(52) U.S. Cl. 424/440; 427/2.14

(57) ABSTRACT

A hard coated confectionary product having a consumable, soft, high solids chewy core fortified with heat sensitive medicaments and vitamins with the core encapsulated in a hard consumable coating and a method for making the fortified confectionary is described.

### FIELD

The present application is generally directed to a hard coated confectionary product having a consumable, fortified, soft chewing core enrobed in a hard consumable coating and a method for making the product. More particularly, the confectionary product has a high solids core which includes a heat sensitive particulate active, such as a vitamin or blends of vitamins and medicaments, surrounded by a sweet hard consumable coating.

### BACKGROUND

Hard coated candy products for delivery of active ingredients are known in the confectionary art. U.S. Patent Publication 2003/0017202 by Bunick et al. describes a coated soft core consumable product with an active that can include vitamins, therapeutics and antacids such as calcium carbonate. If the active has an objectionable taste, it is coated with a taste masking agent (see paragraph 0017). Bunick also describes using excipients (inert substances) in the core where the excipient may include gums (see paragraph 0028). Bunick, focused on the particle size of his active or drug, and the weight ratio of active to shell. Further, he used a taste masking agent to mask off flavors of the active. In short he does not describe a cooked confection having a soft chewy mouth feel or use natural confectionary ingredients, such as gum/emulsifier/fat/coating to deliver an active, such as a vitamin, in a soft chew confectionary product.

U.S. Pat. No. 5,637,313 to Chau et al. describes a soft chewable dosage form which includes a matrix of hydrogenated starch hydrolysate, a water soluble bulking agent and a water insoluble bulking agent. Gums are described as water soluble bulking agents (see column 4, line 67) and calcium carbonate is described as a bulking agent (column 4, lines 21-26). But Chau focuses on an uncooked soft chew product. Moreover, Chau does not use fat, an emulsifier and gum to achieve a high loading of solids which includes calcium carbonate or other antacid. While Chau does contemplate hard coating as an option, Chau does not suggest the use of the hard coating with a cooked confectionary product to stabilize or preclude the confection or active from picking up water and adversely affecting the organoleptic properties of the confectionary product. Moreover, when Chau has been practiced, it has been observed that Chau's uncooked product is extremely soft and not readily formable for subsequent coating.

U.S. Pat. No. 5,013,716 to Cherukuri et al. describes an uncoated confection which includes an active/drug (such as acetaminophen), a gum base which may include calcium carbonate (column 9, line 4) and xanthan gum which may be used as a softener (column 9, line 45). Cherukuri, however, is primarily concerned with a taste masking agent, and does not use the combination of gum/emulsifier/fat/coating to deliver an active, such as an antacid, in a coated confectionary product. Nor does Cherukuri suggest the use of the hard coating with a cooked confectionary product to stabilize or preclude the confection or the antacid from picking up water and adversely affecting the organoleptic properties of the confectionary product.

Heretofore, stable soft chew confectionary delivery systems have been either unknown or difficult to make. Loading heat sensitive actives into a cooked soft chew core has been difficult, especially at high solids levels, because these actives will be deleteriously affected or destroyed by heat at the cooking temperatures used to prepare soft chew cores. This has been a problem with the cooked product and method described in U.S. Patent Publication No. 2009/0011079.

U.S. Pat. No. 6,673,389 to Yang et al. describes an uncoated cooked chewy confectionary. Key to Yang's product is a ratio of reducing to non-reducing sugar. Yang asserts that a conventional ratio of non-reducing to reducing sugars or carbohydrates outside the ratio of reducing to non-reducing sugars (which Yang asserts to be critical) will be sticky or grainy. Reference to Yang's examples show that some ingredients are added with low heat (example 1 “four hundred grams of cocoa butter was heated to 120° F. to 130° F. and 15 g lecithin was added with agitation.”) then cooked at a higher temperature, e.g. 245° F. to 246° F. to form a precooked base to which the calcium carbonate and vitamins were added after only slight cooling. See Yang at column 8, lines 15-59 and column 10, lines 3-22 (Example 1). Yang's low heat mixing with later cooking will affect a crystalline firm product with gritty mouth feel, especially at higher solids levels.

Further soft chew confections, especially those confections which are used for the delivery of active ingredients such as vitamins, analgesics and antipyretics (such as acetaminophen), typically have been hard to stabilize over long periods of time by virtue of their tendency to pick up water under humid conditions or loose water under dry conditions. Not only the active may be hygroscopic, but the confection itself may be hygroscopic due to the high moisture content of chewy confections and high Equilibrium Relative Humidity (ERH) of the confection. As used herein, ERH is the equilibrium state at which the confectionery product neither absorbs nor loses moisture to the environment.

### SUMMARY

Described herein is a shelf life stable coated chewable product having a soft, chewable, cooked, confectionary core with gum, fat, an emulsifier, and a heat sensitive medicament, such as vitamins which are uniformly dispersed in the core. The core is chewy and has a hardness such that it takes less than about 100 Newtons (but more than about 10 N) of force to crack or deform the core at room temperature. In an important aspect, the core comprises at least 10 weight percent, and preferably at least 15 weight percent vitamins, and generally about 15 to about 25 weight percent particulate vitamins. However, other amounts of vitamins or other heat sensitive materials may be included.

The chewable product may be prepared by cooking the core and then subsequently adding and mixing the heat sensitive medicament to the core after cooling the core to at least below 50° C. to form a cooled soft chew core product. Further, the cooled soft chew core product can be precoated with ingredients to provide a moisture barrier that will permit pan coating in a mass production. Throughout the pre-coating process the core maintains a hardness requiring at least 300 N of force to deform the core, yet the core softens after coating to attain the desired chewy texture and mouth feel. Prior to coating, the core will have a hardness such that it takes more than about 300 Newtons (but less than about 600 N) of force to crack or deform the core at room temperature. After coating, the core will cure and soften. After curing, the core will have a hardness such that it takes less than about 100 Newtons (but more than about 10 N) of force to crack or deform the core at room temperature.

In an important aspect, the ingredients which provided a core with such properties are at least about 10 weight percent (dry basis) heat sensitive particulate medicament; about 0.8 to about 2 weight percent (dry basis) gum; about 18 to about 55 weight percent (dry basis) corn syrup; 15 to about 46 weight (dry basis) percent sweetener (other than corn syrup) such as sucrose; from about 0.1 to about 0.5 weight percent (dry basis) emulsifier; from about 3 to 11 weight percent (dry basis) fat and at least about 93% solids. The ingredients, with the exception of the heat sensitive particulate medicament, may be combined with water and then cooked. The cooked mixture may then be cooled to allow the addition of the heat sensitive medicament. Generally, the mixture is cooled to below at least 50° C. before the heat sensitive medicament is added. The heat sensitive medicament may be added, such as by spraying powdered medicaments/vitamins into the cooled coked soft chew confection in multiple streams having a flow rate of about 8-25% dry weight relative to the cooled, cooked mass with a vitamin/medicament particulate size less than about 800 microns, and in some instances less than about 500 microns. The confectionary soft chew core has a hardness such that it takes less than about 100 Newtons (but more than about 10 N) of force to crack or deform the core at room temperature.

The coated cooked core product also provides great versatility and stability in commercial distribution channels because it resists drying out in the core in hot dry conditions and resists taking up water and becoming soft in humid conditions. This is an important problem which has to be addressed for a chewy matrix which is hygroscopic and which product may have a heat sensitive particulate medicament, such as vitamins, which also may be hygroscopic. The edible coating, which encapsulates the cooked, soft, chewable and active fortified confectionary core, provides a chewable product with a highly improved taste profile and also provides the chewable core with organoleptic stability. Improved taste is achieved without special taste masking agents or encapsulating the individual heat sensitive particulate medicament prior to mixing them into what will become an encapsulated core (there is a masking flavor used in the core. As for stability, the coating which encapsulates the cooked core precludes the cooked, soft, vitamin/confectionary core from taking up water or losing water and deleteriously affecting the organoleptic properties of the coated product. The coating is thin and completely surrounds or encapsulates the cooked, soft, active/confectionary core and has a thickness which is effective for protecting the core from a water loss or gain of not more than about 2 weight percent for at least 18 months at ambient temperature and adversely hardening or softening the confectionary core. The coating generally is from about 0.5 to 2.5 mm thick, comprises from about 25 to 45 weight percent of the finished product which is the core encapsulated in the coating, and in an important aspect, the coating comprises substantially crystallized sugar and/or sugar alcohols.

In the core, the gum with curing the coated product keeps the cooked, coated, soft, confectionary core soft and chewy. The combination of gum/emulsifier/fat provides a cooked matrix for the core which keeps the fat within the matrix to deliver a solid, heat sensitive particulate active, such as a heat sensitive vitamin, in a coated confectionary product with a soft non-gritty core. Keeping the fat component with the matrix core keeps the product with a soft and chewy core and organoleptically acceptable. Further, depending upon the atmospheric conditions, the coating protects the confectionary core from drying out resulting in an undesirably gritty product, or alternatively as discussed above, protects the heat sensitive particulate medicament in the core from taking up or losing water and adversely affecting the organoleptic properties of the core, e.g. the core resisting becoming too soft or too hard. The coating also provides a burst of flavor which counteracts a less than desirable taste profile often associated with vitamins and medicaments. In an important aspect, the core is non-aerated or does not have gas blown through it.

In another aspect, a method for making the coated chewable heat sensitive active/vitamin product and loading it with large amounts of heat sensitive vitamins also is described herein. The heat sensitive particulate actives in the product core are added after cooking and cooling with the heat sensitive medicaments/vitamins sprayed via multiple streams into the cooled, cooked soft chew confection and then mixed into the product before extruding and forming the product.

### DESCRIPTION OF THE DRAWINGS

FIGS. 1a and 1b are schematic drawings of the process for making the soft chew core described herein along with the pre-coat addition; and

FIG. 2 is a schematic drawing of the process for coating the core.

### DETAILED DESCRIPTION

The product described herein includes a hard consumable encapsulating coating, such as a sugar coating, and a fortified chewy cooked core. The chewy core will have at least about 10 weight percent (dry basis) particulate active, generally at least 12 and preferably from 15 to 25 weight percent (dry basis), and most preferably about 17 weight percent (dry basis) heat sensitive particulate active; about 0.5 to about 2.5, weight percent (dry basis), and preferably from about 0.8 to about 2 weight percent (dry basis), and most preferably about 1.65 weight percent (dry basis) gum; from about 18 to about 60 weight percent, and preferably from 35 to 38 weight percent (dry basis), corn syrup; from about 15 to about 46 weight percent (dry basis), and preferably from 28 to 32 weight percent (dry basis), sweetener such as sucrose (other than corn syrup); from about 0.10 to about 0.52 weight percent, and preferably from about 0.2 to about 0.4 weight percent (dry basis), most preferably 0.35 weight percent (dry basis), emulsifier; from about 3 to about 11 weight percent (dry basis), and preferably from about 4 to about 8 weight percent (dry basis), most preferably about 7 weight percent (dry basis), fat; and at least about 92 weight percent solids, generally from about 92 to 96 weight percent, and preferably from 93 to 95 weight percent solids.

Further, the cooked core does not require starch or hydrogenated starch hydrolysate to provide a chewable matrix for the core. Heat sensitive particulate vitamins and other medicament actives which may be used as actives in making the chewable product include vitamin E, biotin, folic acid, pantothenic acid, vitamin B, and/or B12 and/or B6, vitamin C, idodine, zinc, choline and inositol. In addition to gum arabic, xanthan gum, carrageenan gum, locust bean gum, karaya gum, acacia gum and guar gum may be used as gums (as opposed to emulsifiers which are not gums) as described herein.

Emulsifiers in addition to gums are used in the composition and process described herein. The amount of emulsifier in the product prevents solids from separating from the product both during making the product with an aqueous slurry and after the product is cooked and extruded. The gum/emulsifier/fat combination provides a matrix so that the fat stays within the matrix and the solids will not separate from the product during and after processing. The emulsifier to fat ratio in the core is from 0.04 to 1.2 on a wet basis. Emulsifiers which may be used in the invention include lecithin, glyceryl monostearate and the like. Texturizing agents may include, by way of example, dextrin, soluble fibers, gelatin and modified food starch. Sugars which may be used include sucrose, glucose, mannose, galactose, ribose, lactose and maltose among others.

As used herein heat sensitive means an active ingredient such as particulate, a vitamin mineral, or medicinal used in the treatment of humans. As used herein, heat sensitive generally means materials that degrade above about 60° C. In one form, heat sensitive materials have 10% or more of the material that degrades above about 60° C. In another form, heat sensitive materials have 5% or more of the material that degrades above about 60° C. In yet another form, heat sensitive materials have 2% or more of the material that degrades above about 60° C.

In one form, the method for manufacturing the product includes adding gum 2 and water 4 to a batching kettle 5 to make a gum/water blend. Next, the gum/water blend is moved from the batching kettle 5 to a weighing vessel 8. Then corn syrup 10, fat 12, a non invert sugar 14, and emulsifier 16 are weighed into vessel 8 and then moved to surge kettle 20. The latter blend of water/gum/corn syrup/sugar/emulsifier ingredients are moved via line 22 from the surge kettle to a cooker 24. At the cooker 24, the ingredients are cooked at from about 118° C. to about 121° C., preferably at about 119° C. to about 120° C. for about 0 to about 30 minutes at atmospheric pressure.

At the start of cooking the aqueous slurry includes from about 18 to about 60 weight percent corn syrup, preferably from about 35 to about 38 weight percent corn syrup, from about 0.10 to about 0.52 weight percent, preferably from about 0.20 to about 0.40 weight percent, emulsifier, from about 3 to about 11 weight percent, and preferably from about 4 to about 8 weight percent, fat, from about 0.5 to about 2.5 weight percent gum, and at least 10 weight percent active, generally from about 12 to about 25 weight percent particulate active, preferably from about 15 to about 25 weight percent particulate active. The percentages in the slurry are on a dry basis and are dry percentages. The slurry comprises at least about 67 weight percent solids, generally from 68 to 84 weight percent, and preferably from about 72 to about 75 weight percent solids, and from about 25 to about 28 weight percent water. Sucrose is a preferred sweetener/sugar. But sugar alcohols may be used as sweeteners in lieu of corn syrup and sugars/carbohydrates. The combination of corn syrup and sweetener should comprise from about 60 to about 80 weight percent (dry basis), and preferably from about 67 to about 72 weight percent (dry basis), of the slurry. Corn syrup comprises from about 40 to about 70 weight percent (dry basis), preferably from about 45 to about 65 weight percent (dry basis) of the corn syrup/sweetener combination.

After the slurry is cooked at about 118° C. to about 121° C., it then is moved via line 26 to a vacuum chamber 28 which has a pressure of about 450 to about 550 mbr. The product temperature is from about 118 to about 121 when it enters the chamber and is held in the chamber for about 0 to about 30 seconds. Thereafter the moisture depleted product for the core is moved via line 30 to mixing tank 32 which mixes the product at a temperature of from about 95° C. to about 105° C. where flavor 34 and color 36 are added to the moisture depleted product.

After cooking and after flavors and colorants are blended and mixed at a temperature of at least about 90° C., and generally from about 95° C. to about 110° C. for about 5 to about 10 minutes with the cooked product in mixer 32, the mixed product mass with flavor and color is conveyed as a viscous mass via line 38 onto a cooling drum 40 having a temperature of from about 14 to about 19° C., and generally about 17.5° C. where the product is converted into a planar web having a temperature of not over about 50° C., and generally about 40° C. to about 45° C. That planar web is conveyed onto a transfer belt 42 to move the product to spray feeder 44 where the product has a temperature of from about 20 to about 50° C.

At the spray feeder the heat sensitive solid particulate active is fed into a vessel holding the cooled, cooked mass as a spray from nozzles above the vessel. More specifically, the heat sensitive solid particulate active is collected in a hopper and then fed through a long tube via a device such as a screw. The long tube has a plurality of openings or holes along the length which allow the heat sensitive solid particulate active to fall onto a conveyor onto the cooled cooked mass as a layer. In one form, the layer of heat sensitive solid particulate active is approximately 1/16 inch thick and extends substantially along the width of the conveyed cooled, cooked mass, approximately ¼ inch thick. In one form, the heat sensitive solid particulate active is applied in a range of about 8-25% dry weight of the cooled, cooked mass. In another form, the heat sensitive solid particulate active is applied in a range of about 17% dry weight of the cooled, cooked mass. In one form, the heat sensitive solid particulate active includes particles which are smaller than 800 microns and in another form, particles which are smaller than 500 microns. The conveyed material is then folded at least once onto itself, such as by using a folding or rolling machine. After folding, the cooled product with active is moved from the spay feeder via line 46 to extruder 48 for further mixing.

The temperature of the entry point of extruder 48 is about 46° C., and in the extruder product with actives undergoes further mixing with shear which keeps the product malleable for subsequent processing, coating and forming. The temperature in the extruder 48 may range between about 30° C. to below about 60° C. and, in one form, the extruder head is set for about 43 to 50° C. and the mass with active is extruded into a rope for further forming.

After extrusion, the product is extruded, it is moved via line 50 to batch former 52 where the product is formed at from about 30 to about 60° C. into a conical shape. It should be noted that the lines, such as line 50 may include heaters and/or coolers to help maintain the product at the desired temperature for forming and to allow the product to properly flow through the equipment (such as below about 60° C. and more preferably below about 50° C.). The latter formed product is moved to sizing roller station 56 via line 54 where it is sized into a rope that is ¼ to ¾ inches in diameter. From sizing roller station 56, the product is moved via line 58 to a forming machine where the product is formed into spherical pieces at a temperature of from about 30 to about 60° C. Thereafter the formed product is moved from the forming machine via line 62 to a sweet cooler 64 where it is cooled to a temperature of from about 10° C. to about 14° C. and then moved via transfer belt 66 to conditioner 68 where the product is held for about 65-75° F. for about 24 hours at a relative humidity of from about 30% to about 35% and preferably for about 18 hours at 70° C. at a relative humidity of about 30. This allows the moisture in the product to equilibrate in the product and allow a later applied precoat barrier to harden providing a moisture barrier and surface texture to accept the final sugar/fat coating. Further, this provides the material time to settle and complete any expansion or contraction to avoid cracking of the precoat and/or coat.

Prior to precoating, but after sizing or pelletizing, the confection has a hardness such that it takes more than about 300 Newtons of force to crack or deform the core at room temperature. After coating this hardness will soften or change as described below.

The cooled sized conditioned product is precoated with an aqueous solution of starch and sugar at prepan 70. In one form, the precoat 0.07-0.12 grams per piece. The precoat is generally about 8-10% of the mass of the finished and coated product. The precoated product also is then further cured at about 70° C. to a relative humidity of less than about 30% to allow the moisture to equilibrate in the product and allow the precoat barrier to harden providing a moisture barrier and surface texture to accept the final sugar/fat/chocolate coating.

The precoated product 80 is conveyed at line 82 to a finishing pan 84. The finishing pan 84 may include an engrossing tank 86 and a smoothing tank 88 whereby a coating is provided from a mixing tank 90. The coating is approximately 0.4 to about 0.7 grams per piece. The coated product is then conveyed at line 92 to a tempering location 94 whereby the product is left set in ambient conditions (68-77° F. and 40-60% relative humidity) until it can be sorted and packaged. The tempered product may then be transferred to a product sorter 96 and optionally a printer 98 before final packaging.

The sugar and sugar alcohol coatings are substantially crystallized after coating. The finished product comprises from about 38 weight percent to about 42 weight percent coating which is in an amount and thickness which are effective for keeping the core from loosing or gaining more than 2 weight percent moisture over a storage period of 18 months at ambient temperature. Generally the coatings are thin, about 0.5 mm to 2.5 mm thick, which thickness is particularly appropriate for a 1.6-1.8 gram finished product comprising 25-45 weight percent coating.

The finished product will also undergo graining whereby sugar crystals, which were dissolved in the matrix during cooking, are permitted to recrystallize. Generally, the ratio of sugar and corn syrup may be used to inhibit graining during processing of the material. In one form, the ratio of sugar to corn syrup will be about 3:2 to about 2:3. In this regard, if the sugar crystals were to prematurely grain, the material may become difficult to process in the machinery. Therefore, it is often desired to slow and/or prevent graining at least until after the material has been extruded.

The hardness of the finished product will change after processing such that the product will become softer and more chewable. For example, immediately after production, the finished product will have a hardness such that it takes more than 300 Newtons of force to crack or deform the core at room temperature. Approximately 10-30 days after completion of the finished product, the hardness of the product is soft enough to be easily consumable, such as requiring less than about 100 Newtons of force to crack or deform the core at room temperature. In one form, after approximately 15-20 days the finished product will have a hardness that is soft enough to be easily consumable.

### Example 1

An exemplary process and combinations of ingredients will be described below. However, it should be understood that this is merely one form and that other forms and combinations are contemplated. For example, one exemplary listing of the inactive ingredients used in one form of the composition is listed below in Table 1.

TABLE 1
Inactive Ingredient List

| Ingredient | Total (g) | PC % |
|---|---|---|
| Granulated Sugar | 0.586 | 37.21% |
| Corn Syrup | 0.383 | 24.32% |
| Sucrose | 0.318 | 20.19% |
| Coconut Oil | 0.074 | 4.70% |
| Water | 0.074 | 4.70% |
| 6x Sugar | 0.038 | 2.41% |
| Instant Gum | 0.031 | 1.97% |
| NF Quick Coat | 0.032 | 2.03% |
| Instant Pure-Cote B792 | 0.011 | 0.70% |
| Titanium Dioxide | 0.003 | 0.19% |
| Color Dispersions (Red, Blue, Purple) | 0.005 | 0.32% |
| Capol 217P | 0.005 | 0.32% |
| N&A Flavoring | 0.01 | 0.63% |
| Lecithin | 0.004 | 0.25% |
| Pregelatinised Starch | 0.001 | 0.06% |
| Total | 1.575 | 100.00% |

The inactive ingredients may be combined with a variety of different heat sensitive particulate medicaments. For example, one form of a combination of vitamins, minerals and nutrients is shown below in Table 2. It should be understood that the heat sensitive particulate medicament may be a single component or a plurality of components, such as shown in Table 2. Furthermore, a variety of different ranges of the components may be included as desired.

TABLE 2
Active Ingredient List

| | | Amount of | |
|---|---|---|---|
| | | Claim | |
| Claim | Ingredient Source | Per Serving | |
| Vitamin A | as Palmitate, USP-FCC | 2000 | IU |
| Vitamin D3 | as Cholecalciferol, USP-FCC | 400 | IU |
| Vitamin E | as dl-alpha-Tocopheryl Acetate, | 20 | IU |
| | USP-FCC | | |
| Biotin | as Biotin-USP | 75 | mcg |
| Folic Acid | as Folic Acid-USP | 0.2 | mg |
| Pantothenic Acid | as Calcium d-Pantothenate, USP-FCC | 5 | mg |
| Vitamin B12 | as Cyanocobalamin, USP | 5 | mcg |
| Vitamin B6 | as Pyridoxine HCI, USP-FCC | 1 | mg |
| Vitamin C | as Ascorbic Acid, USP-FCC | 30 | mg |
| Iodine | as Potassium Iodide, USP-FCC | 40 | mcg |
| Zinc | as Zinc Sulfate, USP-FCC | 2.5 | mg |
| Choline | as Choline Bitartrate, USP-FCC | 38 | mg |
| Inositol | as Meso-Inositol | 20 | mcg |

One form of a combination of ingredients will now be described. For example, a gum solution may be prepared as shown below in Table 3. This gum solution may be prepared separately or may be added to a base recipe for the confectionary core. As shown in Table 3, the gum recipe generally includes water, titanium dioxide and instant gum.

TABLE 3
Gum Solution Recipe

| Ingredient | Wet % | % Solids | PC % | Dry % |
|---|---|---|---|---|
| Potable Water | 51.55% | 0.00% | 51.55% | 0.00% |
| Titanium Dioxide | 7.22% | 99.00% | 7.22% | 16.07% |
| Instant Gum | 41.24% | 90.50% | 41.24% | 83.93% |
| Total | 100.00% | | 100.00% | 100.00% |

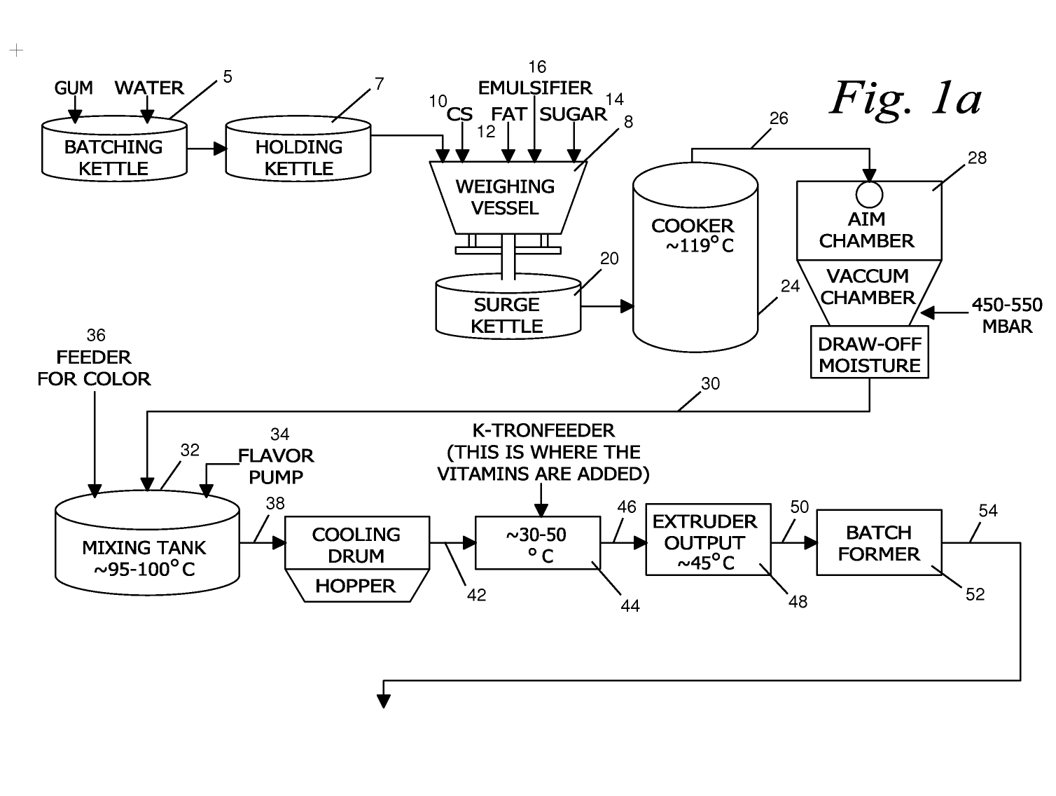

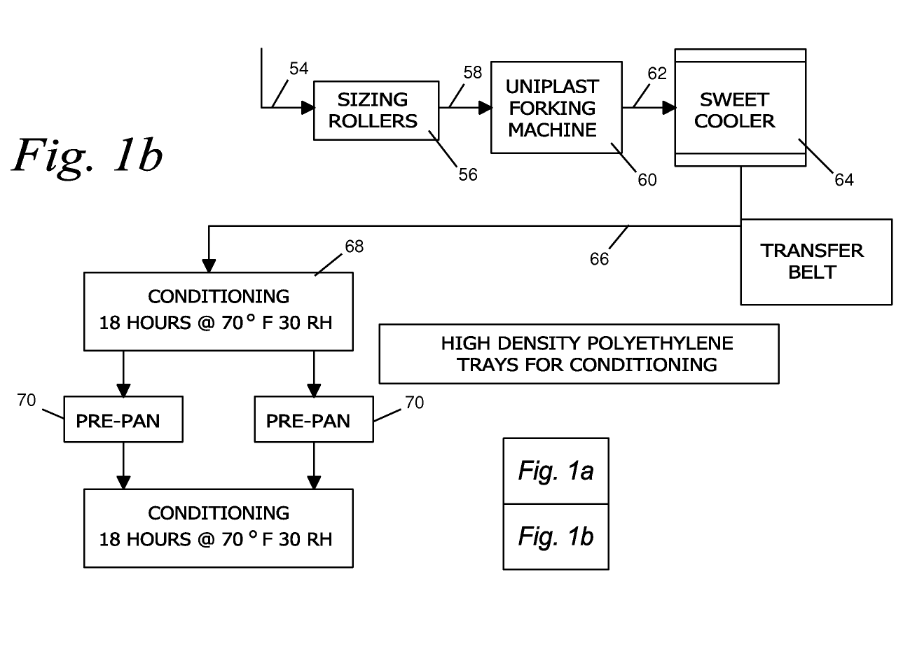

The base recipe for the confectionary core is shown below in Table 4. This base recipe includes the gum solution shown in Table 3. Again, as mentioned above, the gum solution may be a premix that is added to the base recipe or the ingredients of the gum solution may be added individually to the base recipe. The components in the base recipe are then cooked as described above and as illustrated in FIG. 1. Once the ingredients have been cooked, they generally form a cooked syrup for the confectionary core.

TABLE 4
Base Recipe

| Ingredient | Wet % | % Solids | PC % | Dry % |
|---|---|---|---|---|
| Sucrose | 44.23% | 67.00% | 29.64% | 39.83% |
| Corn Syrup | 44.23% | 80.50% | 35.61% | 47.85% |
| Coconut Oil | 6.93% | 100.00% | 6.93% | 9.32% |
| Gum Solution | 4.27% | 44.46% | 1.90% | 2.55% |
| Lecithin | 0.33% | 100.00 | 0.33% | 0.45% |
| water | 0.00% | 0.00% | 25.59% | 0.00% |
| Total | 100.00% | | 100.00% | 100.00% |

The cooked syrup may then be combined with other ingredients, such as flavorings and sweeteners as shown in Table 5 below. Furthermore, the cooked syrup is cooled and then heat sensitive particulate medicaments may be added, such as Multivitamin blend. One form of this blend was described in Table 2. As seen in this table, the cooked syrup is combined with flavorings and sweeteners.

TABLE 5
Cooked syrup with sweeteners, flavoring and heat sensitive particulate
medicaments.

| Ingredient | Belt % | % Solids | PC % | Dry % |
|---|---|---|---|---|
| Cooked Syrup | 78.67% | 92.40% | 72.69% | 77.32% |
| Multivitamin Blend | 17.08% | 100.00% | 17.08% | 18.17% |
| Berry/Masking | 0.60% | 100.00% | 0.60% | 0.64% |
| Flavor Blend | | | | |
| N&A Sweetness | 0.15% | 100.00% | 0.15% | 0.16% |
| Enhancer | | | | |
| 6x Sugar | 3.5% | 99.60% | 3.49% | 3.71% |
| Water | 0.00% | 0.00% | 5.99% | 0.00% |
| Total | 100.00% | | 100.00% | 100.00% |

One form of a flavoring combination is shown in Table 6 below. As shown in this table, the flavoring is for a berry flavor. It should be understood that other flavorings may also be used. Furthermore, the flavoring components may be in the form of a premix added to the composition or the ingredients may be added individually.

TABLE 6
Berry/Masking Flavor Blend

| Ingredient | Wet % | % Solids | PC % | Dry % |
|---|---|---|---|---|
| Forest Berry Flavor | 58.33% | 100.00% | 58.33% | 58.33% |
| Masking Flavor | 41.67% | 100.00% | 41.67% | 41.67% |
| Total | 100.00% | | 100.00% | 100.00% |

A summary of the breakdown of the combination of the core ingredients is illustrated below in Table 7 prior to precoating and coating.

TABLE 7
Core Formula Ingredient Breakdown

| Ingredient | PC % | Dry % |
|---|---|---|
| Water | 5.9927% | 0.0000% |
| Sucrose | 28.9513% | 30.7968% |
| Corn syrup | 34.7847% | 37.0022% |
| Coconut Oil | 6.7731% | 7.2048% |
| Instant Gum | 1.5555% | 1.6547% |
| Titanium Dioxide | 0.2978% | 0.3168% |
| Lecithin | 0.3256% | 0.3464% |
| Multivitamin Blend | 17.0833% | 18.1723% |
| Forest Berry Flavor | 0.3500% | 0.3723% |
| Masking Flavor | 0.2500% | 0.2659% |
| N&A Sweetness Enhancer | 0.1500% | 0.1596% |
| 6x Sugar | 3.4860% | 3.7082% |
| Total | 100.0000% | 100.0000% |

A precoating solution may be prepared as illustrated in Table 8 below. In this form, the precoat solution includes granulated sugar, pregelatinised starch and water. It should be understood that other components may be included and may also be included in different proportions and ratios to provide the desired hardness after precoating.

TABLE 8
Pre-Coat Gum Solution

| Ingredient | Wet % | % solids | PC % | Dry % |
|---|---|---|---|---|
| Granulated Sugar | 43.33% | 99.50% | 43.11% | 89.53% |
| PreGelatinised | 6.00% | 84.00% | 5.04% | 10.47% |
| Starch | | | | |
| Potable Water | 50.67% | 0.00% | 51.85% | 0.00% |
| Total | 100.00% | | 100.00% | 100.00% |

In one form, the precoated core has a compositional breakdown as shown in Table 9 below.

TABLE 9
Precoated Centers

| Ingredient | Wet % | % solids | PC % | Dry % |
|---|---|---|---|---|
| Flavored Core | 91.7033% | 100.00% | 91.70% | 92.3182% |
| Pre-Coat Gum | 1.0928% | 48.15% | 0.53% | 0.5297% |
| Solution | | | | |
| Granulated Sugar | 4.4519% | 99.00% | 4.41% | 4.4369% |
| NF Quick Coat | 2.7521% | 98.00% | 2.70% | 2.7151% |
| PC Water | 0.0000% | 0.00% | 0.67% | 0.0000% |
| Total | | | 100.00% | 100.00% |

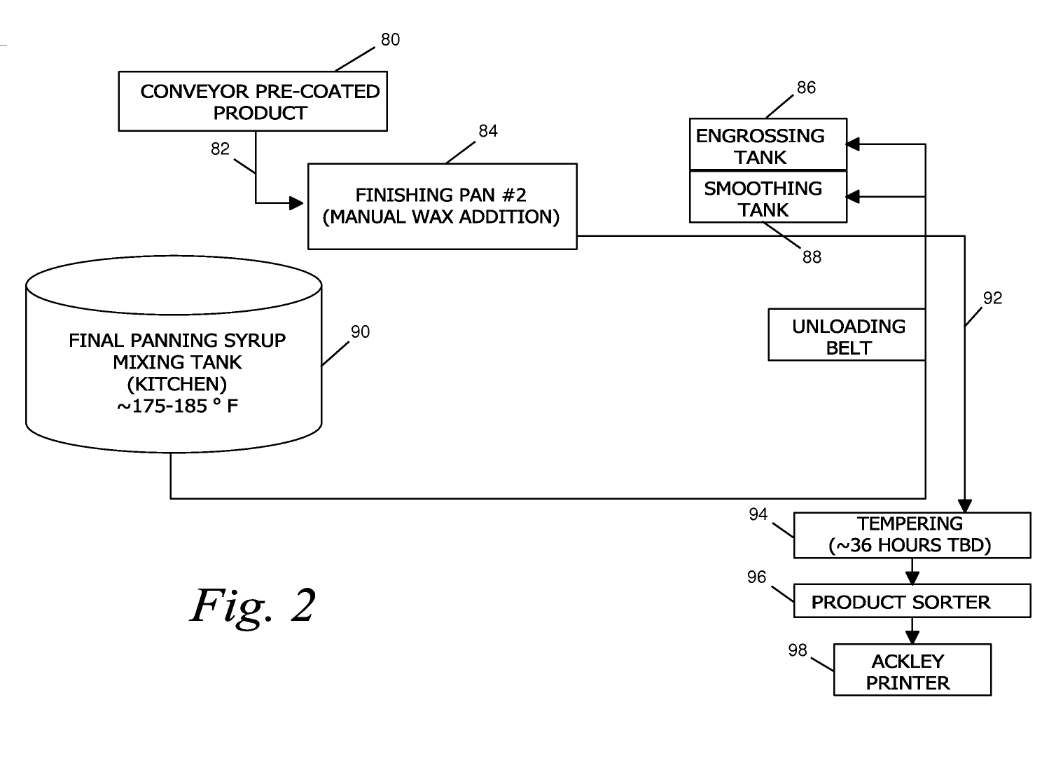

The precoated cores may then be coated as described above with reference to FIG. 2. One form of a formula for the coating process is shown below in Table 10. The composition includes panning syrups which are also described below in Tables 11 and 12.

TABLE 10
Coating Process Per Pan Load

| Ingredient | Wet % | % Solids | PC % | Dry % |
|---|---|---|---|---|
| Pre-Coated Cores | 66.5611% | 100.00% | 73.82% | 73.82% |
| Panning Syrup #1 | 22.8210% | 70.88% | 17.94% | 17.94% |
| Blue Panning Syrup #2 | 10.2359% | 68.90% | 7.82% | 7.82% |
| Blue Raspberry Flavor | 0.1063% | 100.00% | 0.12% | 0.12% |
| Capol 217P | 0.2758% | 100.00% | 0.31% | 0.31% |
| PC Water | 0.00% | 0.00% | 0.00% | 0.00% |
| Total | 100.00% | | 100.00% | 100.00% |

TABLE 11
Panning Syrup #1

| Ingredient | Wet % | % Solids | PC % | Dry % |
|---|---|---|---|---|
| Granulated Sugar | 64.60% | 99.50% | 64.28% | 90.68% |
| Potable Water | 28.10% | 0.00% | 0.00% | 0.00% |
| Instant Pure-Cote B792 | 2.80% | 87.50% | 2.45% | 3.46% |
| Instant Gum | 3.500% | 90.50% | 3.17% | 4.47% |
| (Gum Arabic) | | | | |
| Titanium Dioxide | 1.00% | 99.00% | 0.99% | 1.40% |
| PC Water | 0.0000% | 0.00% | 29.12% | 0.00% |
| Total | 100.00% | | 100.00% | 100.00% |

TABLE 12
Blue Panning Syrup #2

| Ingredient | Wet % | % Solids | PC % | Dry % |
|---|---|---|---|---|
| Granulated Sugar | 66.50% | 99.50% | 66.17% | 96.05% |
| Potable Water | 30.75% | 0.00% | 0.00% | 0.00% |
| Blue Dispersion | 2.75% | 99.00% | 2.72% | 3.95% |
| Post Cooked Water | 0.00% | 0.00% | 31.11% | 0.00% |
| Total | 100.00% | | 100.00% | 100.00% |

A summary of the overall compositional breakdown for one form of the final coated confectionary product including heat sensitive particulate medicaments is shown below in Table 13.

TABLE 13
Overall compositional breakdown for coated confectionary product

| Ingredient | PC % |
|---|---|
| Water | 4.5442% |
| Sucrose | 19.5806% |
| Corn Syrup | 23.5259% |
| Coconut Oil | 4.5808% |
| Instant Gum | 1.9300% |
| Titanium Dioxide | 0.4523% |
| Lecithin | 0.2202% |
| Multivitamin Blend | 11.5539% |
| Flavors | 0.2367% |
| 6x Sugar | 0.1691% |
| Pregelatinised Starch | 0.1014% |
| Granulated Sugar | 2.3577% |
| NF Quick Coat | 0.0406% |
| Instant Pure-Cote B792 | 27.2856% |
| Blue Dispersion | 1.9891% |
| Capol 217P | 0.7024% |
| Total | 100.0000% |

It should be understood that in one form, the amount of each of the ingredients listed above in Table 13 may fall within a range of plus or minus fifty percent. In another form, the amount of each of the ingredients listed above in Table 13 may fall within a range of plus or minus twenty percent.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

1. A confectionary product having a cooked, soft chewy confectionary core with a coated surface selected from the group consisting of substantially crystallized sugar and substantially crystallized sugar alcohols having a melting point of at least about 100° F., the cooked, soft confectionary core comprising:
at least about 10 weight percent (dry basis) heat sensitive particulate medicament;
from about 0.8 to about 2 weight percent (dry basis) gum distributed throughout the core;
from 18 to 55 weight percent (dry basis) corn syrup solids, from 15 to 46 weight percent (dry basis) from a sweetener selected from a group consisting of sugar other than from corn syrup solids, sugar alcohols and mixtures thereof;
from about 0.1 to about 0.5 weight percent (dry basis) emulsifier;
from 3 to 11 weight percent (dry basis) fat;
from 3 to 9 weight percent water; and
at least about 92 weight percent solids in the core, the product having a hardness such that after at least 5 days after production, less than 100 Newtons of force are required to crack or deform the core at room temperature.

2. The confectionary product of claim 1, wherein emulsifier to fat ratio in the core is from about 4 to about 9 weight percent on a dry basis.

3. The confectionary product of claim 2, wherein the core comprises at least 15 weight percent (dry basis) of at least one particulate vitamin, the coating is thin, completely surrounds the core, comprises from about 30 to 50 weight percent of the confectionary and is effective for protecting the core from a water loss or gain of not more than about 2 weight percent for at least 18 months at ambient temperature.

4. The confectionary product of claim 3, wherein at least one vitamin is a vitamin blend.

5. The confectionary product of claim 3, wherein the core is cooked and the vitamins are added to the cooked core at a temperature of from about 30 to about 50° C. and after cooking and coating, the core has an initial hardness such that at least 300 Newtons of force are required to crack or deform the core at room temperature.

6. The confectionary product of claim 3, wherein the core comprises at least 17 weight percent (dry basis) of at least one particulate vitamin.

7. A method for making a fortified chewy confectionary coated with a crystallized coating selected from the group consisting of substantially crystallized sugar and substantially crystallized sugar alcohols having a melting point of at least about 100° F. hard sugar coating, the method comprising:
slurrying corn syrup, fat, a non invert sugar, an emulsifier, gum and water to provide an aqueous slurry having from about from about 0.8 to about 2 weight percent (dry basis) gum, from 18 to 55 weight percent (dry basis) corn syrup solids, from 15 to 46 weight percent (dry basis) non invert sugar, from about 0.1 to about 0.5 weight percent (dry basis) emulsifier, from 3 to 11 weight percent (dry basis) fat, and from 3 to 9 weight percent water;
cooking the aqueous slurry at about 110° C. to about 120° C. effective to remove at least a portion of the water from the slurry to provide a viscous blended product;
cooling the viscous blended product to a temperature of not more than about 50° C.;
blending a heat sensitive particulate medicament at a temperature of not more than 50° C. with the viscous blended product to provide a soft chew core product;
extruding the cooled soft chew core product to provide an extruded product;
forming the extruded product into pieces having a hardness such that at least 300 Newtons of force are required to crack the product at room temperature; and
coating the product with coating to provide a confectionary product having a cooked, soft, chewy confectionary core with a coating which provides a crystallized coating, the confectionary product having a hardness such that after at least 5 days after production, less than 100 Newtons of force are required to crack or deform the core at room temperature.

8. The method of claim 7 further comprising a precoating step prior to the coating step, the precoating step.

9. The method of claim 7, wherein the core is non-aerated and comprises
from 35 to 45 weight percent (dry basis) corn syrup;
from 25 to 35 weight percent (dry basis) of a sugar from other than corn syrup solids; and
from 90 to 97 weight percent solids, and the coating is selected from the group consisting of substantially crystallized sugar and substantially crystallized sugar alcohols, is thin, completely surrounds the core, comprises from about 30 to 50 weight percent of the confectionary and is effective for protecting the core from a water loss or gain of not more than about 2 weight percent for at least 18 months at ambient temperature.

10. A confectionary product having a cooked, soft chewy confectionary core with a hard crystallized sugar coated surface, the cooked, soft confectionary core comprising:
at least about 10 weight percent (dry basis) heat sensitive medicament;
gum;
corn syrup solids;
sweetener selected from a group consisting of sugar other than from corn syrup solids, sugar alcohols and mixtures thereof;
emulsifier;
fat; and
the core having at least about 92 weight percent solids, and the water, the gum, emulsifier and fat in amounts effective to make the cooked core a matrix which keeps the fat within the matrix and permit the core to deliver the heat sensitive medicament as a part of a non-gritty core, the sugar coating having a thickness and being effective for preventing a water loss or gain in the core of not more than 2 weight percent for at least 18 month at ambient temperature, the product having an initial hardness immediately after production such that at least 300 Newtons of force are required to crack the core and a cured hardness such that after at least 5 days after production, less than 100 Newtons of force are required to crack or deform the core at room temperature.

11. The confectionary product of claim 10, wherein the coating comprises from about 30 to about 50 weight percent of the coated confectionary product.
